(19) United States
(12) Patent Application Publication
(10) Pub. No.: US 2009/0062926 A1
(43) Pub. Date: Mar. 5, 2009

(54) Orthopaedic Implants Having Bioresorbable Posts

(75) Inventor: Joseph Wyss, Fort Wayne, IN (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: DePuy Products, Inc., Warsaw, IN (US)

(21) Appl. No.: 12/270,214

(22) Filed: Nov. 13, 2008

Related U.S. Application Data
(63) Continuation of application No. 11/453,745, filed on Jun. 15, 2006.

(51) Int. Cl. A61F 2/30; A61F 2/28; A61B 17/04

(52) U.S. Cl. 623/23.42; 623/23.6; 606/301

(57) ABSTRACT

An orthopaedic implant for replacing the surface of a bone, such as in a joint arthroplasty, includes a bone facing surface for mating with the prepared end of the bone. The surface includes a porous coating to promote bone ingrowth. A bioresorbable post extends from the bone facing surface for engagement within a prepared cavity or bore in the bone. The bioresorbable material of the post is selected so that the post will not be substantially resorbed into the existing bone until substantial bone ingrowth has been achieved in the porous coating.

This application is a continuation of co-pending application Ser. No. 11/453,745, filed on Jun. 15, 2006, the disclosure of which is herein totally incorporated by reference in its entirety.

### BACKGROUND OF THE INVENTION

The present invention concerns orthopaedic implants and more particularly to features of such implants that help preserve the patient's natural bone.

Most joint arthroplasty procedures require replacement of one or more articulating surfaces of the joint with a prosthesis. These prosthesis are typically formed of a biocompatible metal, such as stainless steel or titanium, or occasionally of a ceramic material. The prosthesis or orthopaedic implant must be sufficiently strong to endure what may be very significant loads over a long period of time. For instance, a hip implant will endure cyclic loads in the neighborhood of two or three times the patient's body weight during normal usage of the joint and even greater loads during athletic activity. Consequently, the orthopaedic implant must, by necessity, be formed of a material that can withstand these long-term loading patterns. Moreover, in some instances, the implant must provide a bearing surface that can achieve articulating motion between an opposing natural or prosthetic mating component without pitting, galling or excessive wear. Again, this requirement dictates the type of material that can be effectively used for the orthopaedic implant.

In many cases, the implant material is somewhat incompatible with simple fixation within existing bone. In other words, a porous implant that might integrate well into existing bone may not have the strength and endurance necessary to form a load-bearing component of a joint arthroplasty. Even with a bone cement interface most orthopaedic implants require an additional fixation interface with the existing bone. Thus, many orthopaedic implants rely upon lugs or posts projecting from the bone facing surface of the implant. These lugs or posts are embedded within appropriately formed bores or cavities within the natural bone and fixed, along with the bone facing surface, with bone cement or similar material.

While the use of lugs or posts results in a well-fixed implant, a significant difficulty arises during revision surgery. In general, most joint arthroplasties have a limited life. Prosthetic joint components may loosen over time. In some cases, changes in the patient's joint physiology may render a prosthesis less than optimally suited for the particular joint. In other cases, the natural bone around the implant becomes osteoporotic or may recede from the implant. Sometimes the orthopaedic implant may experience a stress failure during an excessive load event, such as a fall. Thus, many joint arthroplasties will require revision, meaning that the original joint implant is removed and replaced with a new implant component.

When the implant includes lugs or posts that extend into the natural bone, a revision surgery usually requires a large resection of the bone in order to remove the lugs/posts. This large resection not only complicates the surgery, it also requires removal of more of the patient's natural bone than is desirable. This removal of additional bone may further compromise the bone, increase the risk of onset of bone pathologies or abnormalities, or reduce the available healthy bone for fixation of the revision implant. Moreover, the large resection usually means that a larger orthopaedic implant is necessary to fill the space and restore the joint component to its expected geometry.

Consequently, there is a significant need for an orthopaedic implant that can achieve adequate fixation within a patient's natural bone while reducing or eliminating the detriments associated with a subsequent revision surgery.

### SUMMARY OF THE INVENTION

In order to address this need, the present invention contemplates an orthopaedic implant for mounting on the prepared end of a bone which comprises a body having a bone facing surface configured to mate with the prepared end of the bone. In one feature, the bone facing surface is provided with a porous coating adapted for in situ bone ingrowth when the body is mated with the bone. In a further feature, the implant comprises a bioresorbable post extending from the bone facing surface and adapted to extend into a cavity formed in the prepared end of the bone. Thus, the post initially augments the fixation of the implant to the prepared end of the bone.

In one aspect, the post is formed of a bioresorbable material that is selected so that its rate of resorption into the native bone is calibrated relative to the rate of bone ingrowth into the porous coating. In other words, the bioresorbable post remains viable until the bone ingrowth into the porous surface of the implant is sufficient to solidly fix the implant to the bone.

In one feature, the implant includes an engagement feature between the body and the post. In one embodiment, the engagement feature includes a threaded stem on the post and a complementary threaded feature at the bone facing surface of the body. The complementary threads may be formed in a boss projecting from the surface or in a threaded bore defined in the body, depending upon the type of implant. It is preferred that the threaded stem be formed of the same bioresorbable material as the remainder of the post.

In another feature of certain embodiments, the post includes an elongated stem and an external feature to prevent expulsion of the post from the prepared bore in the bone. This external feature may include a number of fins spaced along the length of the post. In some embodiments, the number of fins extend continuously circumferentially around the elongated stem, while in other embodiments, the fins are discontinuous or in the form of circumferential segments. In other embodiments, the fins include a notched surface facing the direction of expulsion of the post.

It is contemplated that the features of this invention may be incorporated into a wide range of orthopaedic implants. The invention is especially valuable for surface replacement implants, such as components of a joint arthroplasty. Thus, the features of the present invention may be incorporated into a femoral, humeral or tibial surface replacement prosthesis.

It is one object of the invention to provide an orthopaedic implant that simplifies a potential revision surgery. More particularly, it is an object to provide an implant that may be removed in a revision surgery without requiring unnecessary removal of additional bone beneath the implant.

Thus, one important benefit of the invention is that it provides an orthopaedic implant that can achieve rigid fixation to a prepared bone surface without the usual difficulties in a later revision procedure. Other objects and benefits of the invention will be appreciated from the following written description and accompanying figures.

### DESCRIPTION OF THE FIGURES

FIG. 1 is a bottom perspective view of a femoral surface orthopaedic implant incorporating the features of the present invention, including a bioresorbable post of the invention.

FIG. 2 is a bottom perspective view of an alternative femoral surface orthopaedic implant incorporating two bioresorbable posts in accordance with features of the present invention.

FIG. 3 is a front perspective view of a bioresorbable post according to one embodiment of the invention, capable of use with orthopaedic implants such as the implants shown in FIGS. 1-2.

FIG. 4 is a front perspective view of a bioresorbable post according to a further embodiment of the invention, capable of use with orthopaedic implants such as the implants shown in FIGS. 1-2.

FIG. 5 is an enlarged view of a notched fin modification to the post shown in FIG. 4.

FIG. 6 is a front perspective view of a bioresorbable post according to a yet another embodiment of the invention, capable of use with orthopaedic implants such as the implants shown in FIGS. 1-2.

FIG. 7 is a side partial cross-sectional view of a hip surface orthopaedic implant incorporating a bioresorbable post in accordance with features of the present invention.

FIG. 8 is a side partial cross-sectional view of a humeral surface orthopaedic implant incorporating a bioresorbable post in accordance with features of the present invention.

FIG. 9 is a bottom perspective view of a tibial surface orthopaedic implant incorporating two bioresorbable posts in accordance with features of the present invention.

FIG. 10 is a cross-sectional view of an implant in accordance with one embodiment of the invention fixed within a bone and illustrating the cut line for a revision procedure to remove the surface implant.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

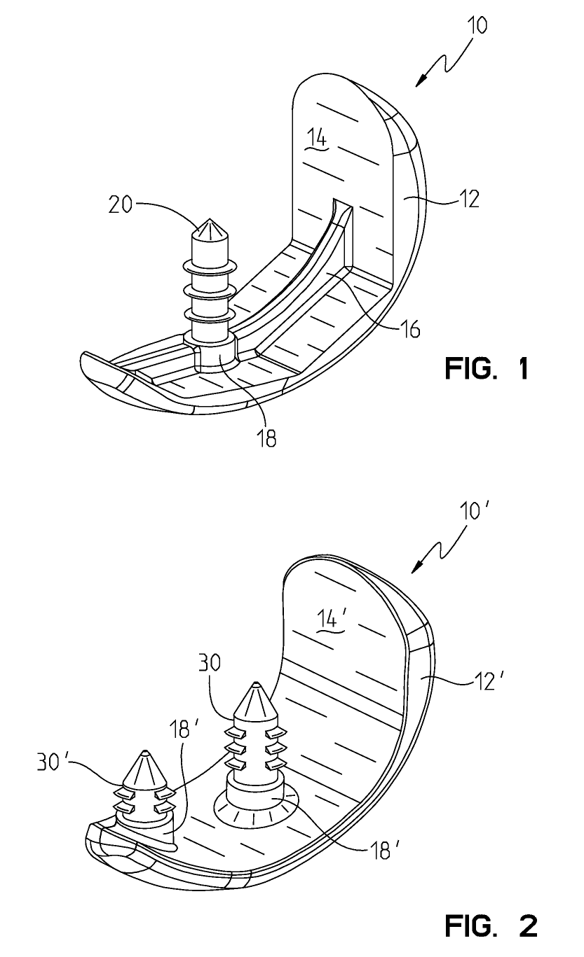

The present invention contemplates an orthopaedic implant, especially an implant configured to replace an articulating joint surface. Thus, in one embodiment, a femoral implant 10 shown in FIG. 1 is provided with a body 12 that is configured to replace the distal surface of the femur as part of a knee joint arthroplasty. The body is formed of a known implant material, and preferably a metallic material such as steel or titanium and alloys thereof. For some implants, the body may be formed of a biocompatible ceramic. The body 12 includes a bone facing surface 14 that is configured to mate with a distal end of a femur prepared in accordance with known practice. The bone facing surface 14 may further define a rib 16 that is positioned within a groove defined in the distal end of the femur.

In accordance with one aspect of the invention, the facing surface 14 may be provided with a porous coating 15 (FIG. 10) that is adapted to facilitate and/or promote bone ingrowth. The porous coating enhances the fixation of the implant to the existing bone as the bony scaffolding integrates with the porous coating. The porous coating may be any suitable composition that may be affixed to, bonded to or formed on the bone facing surface 14 of the implant body 12. The bone facing surface may itself be mechanically treated to form a porous array of open cells.

In one specific embodiment, the surface 14 is coated in a known manner and to an acceptable thickness with a porous material such as POROCOAT® porous coating, marketed by DePuy Inc., that incorporates a three-dimensional array of sintered metal alloy beads (such as cobalt-chrome or titanium). This three-dimensional array provides interconnected interstices or pores that allow bone ingrowth in and through the surface. A 0.030-0.035 inch thick layer of POROCOAT® coating having an average pore dimension of about 250 microns has been found to provide a strong implant-to-bone bond for cementless fixation of orthopaedic implants. Other types of suitable surface coatings may be applied to the implant body in a known manner, such as by plasma spraying. The porous coating for the surface 14 may be impregnated with a bone growth facilitating or encouraging substance, including certain proteins, such as bone morphogenetic proteins, demineralized bone matrix, hydroxyapatite and the like.

Even with the use of bone cement to affix the implant 10 to the prepared distal end of the femur, it is known that several weeks are required for proper fixation. In order to enhance the fixation, plugs or posts are typically provided on the bone facing surface of an orthopaedic implant. Thus, in the illustrated embodiment of FIG. 1, a post 20 is provided; however, unlike the posts of prior implants, the post 20 is formed of a bioresorbable material. The bone facing surface 14 of the implant 10 defines an engagement boss 18 that is configured to receive the post 20 is solid engagement.

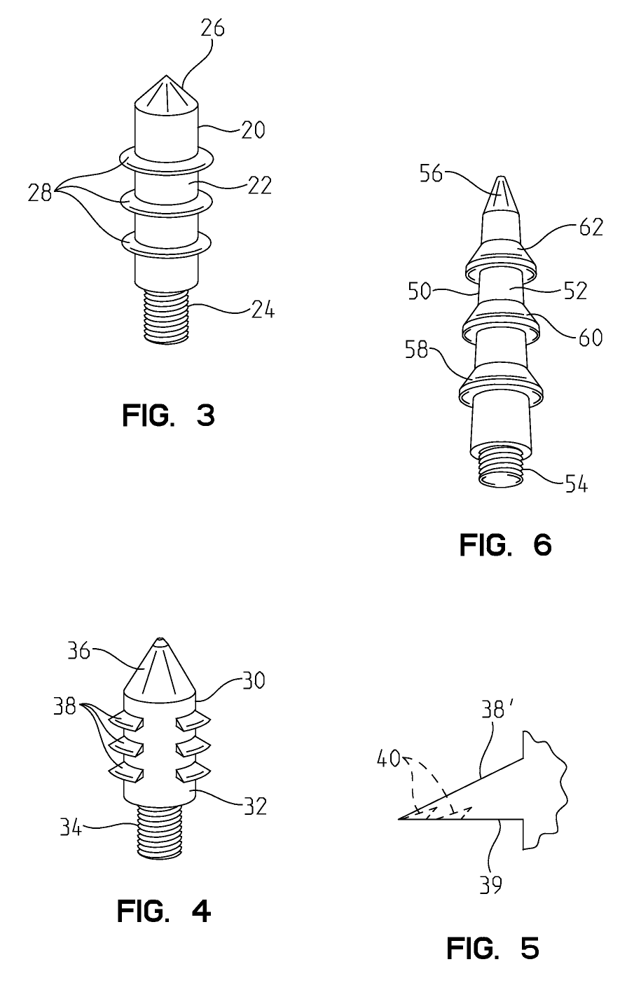

Turning to FIG. 3, one embodiment of the post 20 is illustrated. The post 20 includes an elongated stem 22 that is sized according to the bone in which the post is to be fixed. In particular, the length and diameter of the stem 22 is determined by the available underlying natural bone. The stem 22 is preferably sized in accordance with the plugs or posts incorporated into prior similar orthopaedic implants. Alternatively, the stem 22 may be smaller than the typical posts of the prior art because the porosity of the post 20 may naturally increase the effective surface are of contact between the post and the natural bone.

The bioresorbable post 20 further includes a fixation stem 24, which in the illustrated embodiment is configured with external threads. The boss 18 of the implant 10 thus includes complementary internal threads to mate with the threaded fixation stem 24. Other forms of fixation are contemplated provided that the post 20 is sufficiently rigidly fixed to the body 12 to avoid separation of the components under anticipated joint loading.

The tip 26 of the step may be tapered to facilitate introduction of the post into a prepared bore in the patient's natural bone. The tapered tip 26 is preferably configured to conform to the tapered base of the prepared bore that typically results when the bore is formed by a bone drill.

In order to enhance the fixation of the post 20 within the prepared bore, the stem 22 may incorporate a series of ridges or fins 28 projecting outward from the surface of the stem, as shown in FIG. 3. When the implant 10 is fixed to the distal femur, these fins 28 are preferably embedded within the natural bone surrounding the prepared bore to prevent expulsion or retrograde movement of the post 20 relative to the bone. In one embodiment, the fins 28 have a generally triangular cross-section, as represented in FIG. 5. However, other fin configurations are contemplated that permit introduction of the post into a prepared bone bore and that are able to extend outward into the bone once the post is in position. For example, in one alternative the fins are generally disc-shaped.

The post 20 is formed of a bioresorbable material that is strong enough to firmly engage the natural bone and assist in retaining the implant 10 on the bone. The length of time for the post material to resorb into the adjacent bone is calibrated relative to the bone ingrowth rate into the porous coating of the bone facing surface 14 of the implant 10. In other words, the post preferably remains viable so long as the bone ingrowth is incomplete. Once the bone has fully integrated with the implant 10 through the porous coating, the structure provided by the post 20 is no longer necessary. At this time, the post may be fully resorbed into the existing natural bone.

In one aspect, the entire post 20 is formed of a resorbable material, including the engagement stem 24. Thus, when the post is fully resorbed into the existing bone, a portion of the bone will extend into the engagement boss 18. The interior of the boss may be provided with a bone ingrowth coating to enhance the fixation of the newly formed bone within the boss.

The post 20 is formed of bioresorbable materials that a known to exhibit sufficient strength in orthopaedic fixation applications. For example, suitable resorbable materials include certain polymeric materials, such as poly-alpha-hydroxy acids, poly-L-lactic acid (PLA), polyglactin acid (PGA) and derivatives or composites thereof. The post material may be impregnated with adjunct bioactive compositions to promote bone growth, healing and/or mineralization, including antibiotics, growth factors, bone morphogenetic proteins and the like. As indicated above, the selection of the bioresorbable material is preferably based on the desired resorption or degradation rate in relation to the bone ingrowth rate for the porous coating on the implant 10. Thus, the selected material may have a resorption rate measured in weeks or months, and even up to two years.

In one exemplary embodiment, the post 20 is formed of an injection molded polymer, such as polydioxanone (PDA). The PDA material is completely resorbed in about 210 days (thirty weeks), and is at 71% strength in about 42 days (six weeks). A typical porous coating 15 may achieve substantially complete bone ingrowth in twelve to fifteen weeks, and permanent fixation in about twenty weeks. The post may incorporate an additional resorbable layer over the PDA molded form. For instance, a scaffold of extruded polymer fibers, such as polyglycolic acid/polylactic acid (PGA/PLA) may be fastened to the surface of the post. This PGA/PLA layer will typically have a much faster resorption rate, being fully resorbed in about ten weeks. It is further contemplated that the post 20 itself may be formed entirely of PGA/PLA where a much shorter resorption time is desired in relation to the bone ingrowth rate for the porous coating 15 of the surface implant.

In another embodiment of the invention, a femoral surface replacement implant 10′ includes a body 12′ that incorporates two bosses 18′ on the bone facing surface 14′, as shown in FIG. 2. The bosses 18′ are each configured to receive a post, such as post 30. Post 30 includes an elongated stem 32 with an engagement stem 34, as shown in FIG. 4. The engagement stem 34 may incorporate threads or other forms of engagement with the complementary configured bosses 18′, as in the embodiment of FIG. 3 discussed above. As with the prior embodiment, the length and diameter of the stem 32 is determined by the available bone at the prepared distal end of the femur. As illustrated in FIG. 2, one post 30 is centrally situated on the facing surface 14′, while a second post 30′ is offset. The second post 30′ is shorter than the central post 30 because there is less available natural bone at that location to receive the post.

Returning to FIG. 4, the post 30 includes a tapered tip 36 and several rows of fin segments 38. The segments 38 are in lieu of the circumferential fins 28 of the post 20 in FIG. 3. The use of fin segments, rather than continuous circumferential fins, may make introduction of the post 30 into the prepared bone bore easier.

In one specific embodiment, the continuous fins 28 and the fin segments 38 may be essentially solid. Alternatively, as shown in FIG. 5, the fin segments 38 (and continuous fins) may incorporate notches 40 at the distal face 39 of the segments. The notches 40 face toward the direction of expulsion of the post 30 (and post 20) so that bone may be captured within the notches.

The resorbable posts of the present invention may have a range of configurations that are capable of introduction of the post into a prepared bore and that are adapted to resist expulsion or retrograde movement of the post within the natural bone. Thus, in a further embodiment, a post 50 includes an elongated stem 52 with an engagement stem 54 and a tapered tip 56, as shown in FIG. 6. The stem 52 in this embodiment is tapered toward the tip 56. Rows of circumferential fins 58, 60 and 62 are defined on the stem 52. As with the previous embodiments, the fins 58, 60, 62 are configured to facilitate introduction into the bone bore while resisting expulsion. The fins follow the tapered stem 52 so that each successive fin has a smaller diameter than the last.

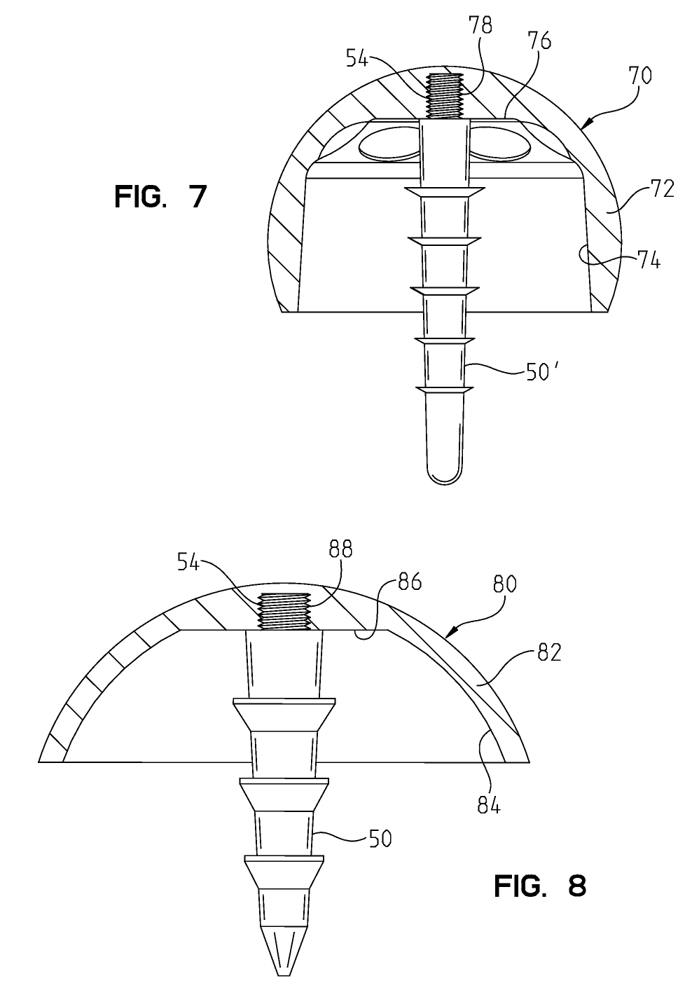

It is contemplated that the resorbable posts and porous surfaces described in connection with the femoral surface replacement implants 10 and 10′ may be incorporated into other types of orthopaedic implants. Thus, as shown in FIG. 7, a hip surface replacement implant 70 includes a generally spherical cup body 72 defining a cavity 74 that conforms to the prepared proximal end of the femur as part of a hip joint arthroplasty. The proximal inner face 76 of the cup body defines a generally central engagement bore 78 to receive the engagement stem 54 of a post 50′. The post 50′ is similar to the post 50 depicted in FIG. 6, but is longer to extend along the intramedullary canal of the femur.

A humeral surface replacement implant 80 is shown in FIG. 8 that is similar in overall construction to the femoral implant. The humeral implant 80 includes a cup body 82 that defines a generally spherical cavity 84 to fit over the prepared proximal end of the humerus as part of a shoulder arthroplasty. The proximal inner face 86 defines an engagement bore 88 for receiving the engagement stem 54 of the bioresorbable post 50 of FIG. 6.

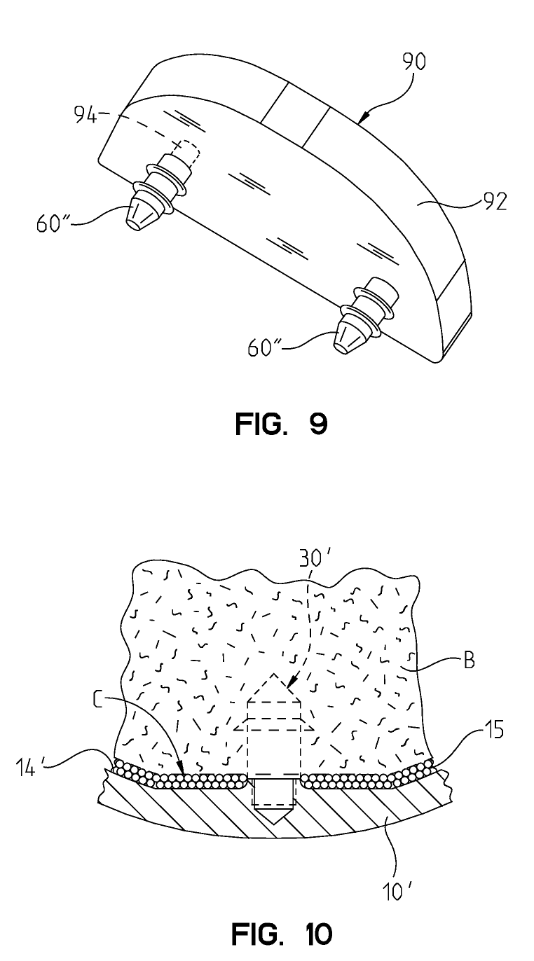

Similarly, the tibial surface replacement implant 90 shown in FIG. 9 includes a body 92 configured as a tibial tray for a knee arthroplasty. The body 92 defines a pair of engagement bores 94 for receiving the engagement stems of resorbable posts 60″. It can be appreciated that the posts 60″ may be similar to any of the posts 20, 30 or 60 shown in FIGS. 3-6.

It is contemplated that the engagement stem 24, 34, 54 on each of the bioresorbable posts 20, 30, 50 allows a particular post to be selected during the arthroplasty procedure. As part of the procedure, the natural bone available for receiving the implant is evaluated, and particularly the available bone for the bore or cavity prepared to receive the bioresorbable post. The length and diameter of the bore is chosen based on the available bone and the selection of available sizes of posts. Alternatively, the post could be specially fabricated to match the prepared bore or cavity.

Once the bioresorbable post has been selected it is engaged to the implant at an engagement boss, such as the bosses 18 or 18′, or engagement bores, such as the bores 78 or 88. The resulting implant is then mounted to the prepared end of the bone according to the particular arthroplasty procedure.

It is contemplated that a kit may be provided in connection with a particular orthopaedic surface implant. The kit may include a selection of posts having different lengths, diameters, number and form of fins, and material resorption rates. The selection of the appropriate post(s) to be engaged to the surface implant may be determined by the nature and extent of available bone for anchoring the implant, the age and health of the patient (as it may affect rate of healing), the need for auxiliary forms of fixation such as bone cement, the type of surface implant being anchored and the composition of the porous coating on the implant.

One important benefit of the present invention may be appreciated upon consideration of FIG. 10. A surface implant, such as the implant 10′ is anchored to the prepared end of a bone B. The porous coating 15 is exaggerated in the view but indicates the region of bone ingrowth between the natural bone B and the facing surface 14′ of the implant. The post 30′ is depicted in phantom to represent that the post has been fully resorbed into the existing bone. In a revision surgery, it can be seen that the cut line C need only follow the facing surface 14′ since the post 30′ is no longer viable. Thus, rather than removing a large portion of the bone B to the depth of the post 30′, the only bone that must be removed is immediately adjacent the implant. Once the original implant is removed, the prepared end of the bone will require only minimal reconditioning to accept a new implant. Moreover, the judicious resection of the bone to remove the original implant means that the new implant can be substantially similar to the original implant, differing primarily in thickness.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

For instance, the porous layers for the bone facing surfaces of each of the implants may incorporate mechanical surface features, such as grooves or “waffling”. The surface features may be used to trap bone cement for fixing the implant to the prepared surface of the natural bone.

What is claimed is:

1. An orthopaedic implant for mounting on the prepared end of a bone comprising:
a body having a bone facing surface configured to mate with the prepared end of the bone;
a porous coating on said bone facing surface adapted for in situ bone ingrowth when said body is mated with the bone; and
a post extending from said bone facing surface and adapted to extend into a cavity formed in the prepared end of the bone, said post formed of a bioresorbable material.

2. The orthopaedic implant of claim 1, further comprising an engagement feature between said body and said post.

3. The orthopaedic implant of claim 2, wherein said engagement feature includes a threaded stem on said post and a complementary threaded feature at said bone facing surface of said body.

4. The orthopaedic implant of claim 3, wherein said threaded stem is formed of said bioresorbable material.

5. The orthopaedic implant of claim 1, wherein said post includes an elongated stem and an external feature to prevent expulsion of said post from the prepared bore.

6. The orthopaedic implant of claim 5, wherein said external feature includes a number of fins.

7. The orthopaedic implant of claim 6, wherein said number of fins extends circumferentially around said elongated stem.

8. The orthopaedic implant of claim 7, wherein said number of fins extend continuous around said elongated stem.

9. The orthopaedic implant of claim 7, wherein said number of fins include a notched surface facing the direction of expulsion of said post.

10. The orthopaedic implant of claim 1, wherein the bioresorbable material is selected so that said post will not be substantially resorbed into the existing bone until substantial bone ingrowth has been achieved in the porous coating.

11. A kit for fixation of an orthopaedic surface implant to a prepared end of a bone, comprising:
a surface implant including;
a bone facing surface configured to mate with the prepared end of the bone;
a porous coating on said bone facing surface adapted for in situ bone ingrowth when said surface implant is mated with the bone; and
at least one engagement feature on said bone facing surface; and
a plurality of differently configured bioresorbable posts, each of said posts including an mating feature for engagement with said engagement feature of said surface implant to engage the post thereto, one of said plurality of posts selectable to engage a corresponding one of said at least one engagement feature when said surface implant is mated with the bone.

12. The kit of claim 11, wherein said plurality of bioresorbable posts includes a plurality of posts having different lengths.

13. The kit of claim 11, wherein said plurality of bioresorbable posts includes a plurality of posts having different diameters.

14. The kit of claim 11, wherein said plurality of bioresorbable posts includes a plurality of posts formed of bioresorbable materials having different resorption rates.
